(19) United States
(12) Patent Application Publication
(10) Pub. No.: US 2024/0067591 A1
(43) Pub. Date: Feb. 29, 2024

(54) Method and system for producing hexafluoro-1,3-butadiene

(71) Applicants: ZHEJIANG RESEARCH INSTITUTE OF CHEMICAL INDUSTRY CO., LTD., Hangzhou (CN); SINOCHEM LANTIAN CO., LTD., Hangzhou (CN)

(72) Inventors: Wucan LIU, Hangzhou (CN); Haifeng WU, Hangzhou (CN); Ling LI, Hangzhou (CN); Hao CHENG, Hangzhou (CN); Jingtian ZHANG, Hangzhou (CN); Jiexun CHEN, Hangzhou (CN)

(21) Appl. No.: 18/114,276

(22) Filed: Feb. 27, 2023

(30) Foreign Application Priority Data
Aug. 18, 2022 (CN) 202210994737.4

(51) Int. Cl. C07C 17/281; B01J 20/20; B01J 37/18; C07C 17/06; C07C 17/23

(52) U.S. Cl. CPC C07C 17/281; B01J 20/20; B01J 37/18; C07C 17/06; C07C 17/23; B01J 23/462

(57) ABSTRACT

Disclosed in the present disclosure are a method and system for producing hexafluoro-1,3-butadiene. It includes: under the action of a catalyst, chlorotrifluoroethylene reacting with hydrogen gas in a first reactor to obtain a mixture, the mixture entering a rectification apparatus, trifluoroethylene obtained by rectification entering a second reactor and reacting with bromine under light to obtain 1,2-dibromo-trifluoroethane; in a third reactor pre-loaded with the 1,2-dibromo-trifluoroethane, adding the 1,2-dibromo-trifluoroethane and solid alkali, and performing reaction to obtain bromotrifluoroethylene; and adding the bromotrifluoroethylene to a fourth reactor holding with zinc powder, an initiator and an organic solvent for reaction, so as to obtain a trifluoroethenyl zinc bromide solution, performing filtration, and then adding a coupling agent for a coupling reaction, so as to obtain hexafluoro-1,3-butadiene. The present disclosure has the advantages of high safety, good in catalytic stability and high in process selectivity, and can achieve continuous production.

### TECHNICAL FIELD

The present disclosure relates to the preparation of fluorine-containing olefin, and in particular, to a method for safely producing trifluoroethylene, and a method and system for producing hexafluoro-1,3-butadiene by means of bromination of trifluoroethylene, dehydrobromination with a solid alkali, zinc insertion reaction and coupling reaction.

### BACKGROUND

Hexafluoro-1,3-butadiene is a dry-etching gas with excellent performance, which is mainly applied to plasma dielectric etching of semiconductors. The hexafluoro-1,3-butadiene has excellent environment performance and etching performance. The ODP of the hexafluoro-1,3-butadiene is 0, GWP₁₀₀ is 290, and the hexafluoro-1,3-butadiene exists in the atmosphere for only 1.9d. Compared with traditional electron etching gases carbon tetrafluoride (CF₄), hexafluoroethane (C₂F₆), perfluoropropane (C₃F₈), octafluorocyclobutane (c-C4F8) and nitrogen trifluoride (NF₃), the hexafluoro-1,3-butadiene has faster etching rate, higher etching selection and higher aspect ratio.

In a process of preparing the hexafluoro-1,3-butadiene, most commonly, for example, disclosed in CN101031529A and CN110590495A, chlorotrifluoroethylene is used as a raw material, and the hexafluoro-1,3-butadiene is prepared by means of hydrodechlorination, bromination, dehydrobromination, preparation of a zinc reagent, and coupling of the zinc reagent. The process route is easy to obtain raw materials and mild in condition, such that the process route is most suitable for industrialization application. However, in actual production, it is found that, 1) the trifluoroethylene is prone to a disproportionated reaction, and prone to disproportionation explosion especially when coexisting with hydrogen, such that there are large safety hazards; and 2) during dehydrobromination of liquid alkali, bromotrifluoroethylene is low in yield and unstable.

For the safety of the trifluoroethylene, a document (Trifluoroethy-lene deflagration, Andrew E. Feiring; Jon D. Hulburt, American Chemical Society: Chemical & Engineering Safety Letters) published by DuPont in 1997 points out that, the trifluoroethylene is subjected to the disproportionated reaction, a lot of heat is released, resulting in increasing of pressure, even explosion. Lisochkin et al (Explosive-hazard estimates for several fluorine-containing monomers and their mixtures, based on the minimum ignition pressure with a fixed igniter energy, Combustion, Explosion and Shock Waves volume 42, pages 140-143 (2006)) point out that, when the trifluoroethylene coexists with the hydrogen, the disproportionation explosion is more likely to occur and releases a lot of heat. Currently, safety studies on the trifluoroethylene mainly focus on purification, transportation and blends, but not on the safety of a production process of the trifluoroethylene; and the implementation of reaction safety of a step of preparing the trifluoroethylene by means of hydrodechlorination of 1,1,2-chlorotrifluoroethylene directly decides the industrialization prospects and market scale of hexafluoro-1,3-butadiene.

For the dehydrobromination of liquid alkali, using an aqueous solution of sodium hydroxide or potassium hydroxide or an alcohol solution to eliminate hydrogen halide is one of the common methods for preparing olefin. For example, disclosed in Patent CN104844411A is to, in a 10% sodium hydroxide solution, prepare bromotrifluoroethylene by means of performing dehydrobromination on 1,2-dibromo-1,1,2-trifluoroethane. For another example, disclosed in Patent CN107032946A is to, under the action of a phase transfer catalyst, use an aqueous solution of sodium hydroxide or potassium hydroxide or an alcohol solution to produce bromotrifluoroethylene by means of performing a dehydrobromination reaction on 1,2-dibromo-1,1,2-trifluoroethane. The preparation of the bromotrifluoroethylene by means of performing dehydrobromination on the 1,2-dibromo-1,1,2-trifluoroethane is a key step for restricting the synthesis yield of the hexafluoro-1,3-butadiene. A lye dehydrobromination reaction has problems of being low in yield, long in reaction time and low in efficiency, and has a large amount of the three wastes, severely restricting the production cost and scale of the hexafluoro-1,3-butadiene, thereby finally affecting the market competitiveness of hexafluoro-1,3-butadiene products. Therefore, it is necessary to develop a new method for preparing bromotrifluoroethylene by means of dehydrobromination of 1,2-dibromo-1,1,2-trifluoroethane.

### SUMMARY

In order to solve the above technical problems, the present disclosure provides a method and system for producing hexafluoro-1,3-butadiene which is high in production safety, high in total product yield and suitable for industrialized production.

A first portion of the present disclosure provides a method for safely continuously producing trifluoroethylene, which is implemented by means of the following technical solution.

Provided is a method for continuously producing trifluoroethylene. The continuous production method includes: under the action of a supported metal nano catalyst, chlorotrifluoroethylene and hydrogen gas being subjected to a hydrodechlorination reaction in a first reactor, so as to obtain a mixture. The mixture includes 0.8%-2.0% of 1,2-dichlorotrifluoroethane (HCFC-123a) and/or 1-chloro-1,2,2-trifluoroethane (HCFC-133). The supported metal nano catalyst includes a first component selected from at least one of ruthenium, palladium or platinum, a second component selected from at least one of copper, bismuth or cerium, and an activated carbon carrier.

Further, the mixture further includes: 20%-50% of trifluoroethylene, 43%-77% of chlorotrifluoroethylene, and 2%-5% of 1,1,2-trifluoroethane (HFC-143).

The temperature of a hydrodechlorination reaction with the chlorotrifluoroethylene and the hydrogen gas is 100-200° C., and a reaction pressure is 0-2 MPa. The total volume space velocity of the hydrogen gas and the chlorotrifluoroethylene is 200-500 h⁻¹; and the mole ratio of the hydrogen gas and the chlorotrifluoroethylene is (1.2-2.5):1.

In the supported metal nano catalyst of the present disclosure, a supported amount is based on the mass of a carrier in the catalyst. the supported amount of the first component is 0.05%-5.0%, and the supported amount of the second component is 0.01%-3.0%; and preferably, the supported amount of the first component is 0.1%-3.0%, and the supported amount of the second component is 0.01%-2.0%.

Further, the first component and the second component meet the following requirements: the mass ratio of the first component to the second component is 1:(0.1-5). Preferably, the mass ratio of the first component to the second component is 1:(0.1-2).

The activated carbon carrier of the present disclosure is granular activated carbon or columnar activated carbon, and a material of the activated carbon carrier is selected from coconut shell, wood or coal activated carbon. Preferably, the specific surface area of the activated carbon carrier is ≥1000 m²/g, and ash content is 3.0 wt %. More preferably, the specific surface area of the activated carbon carrier is ≥1100 m²/g, and ash content is ≤2.8 wt %.

A particle size of the supported metal nano catalyst of the present disclosure is 2-50 nm, and metal particles with particle sizes being 2-10 nm account for more than 90%, such that uniform distribution of the particle sizes is achieved. The specific particle size is calculated by means of the following method: randomly selecting two to three areas in a picture of a Transmission Electron Microscope (TEM), performing amplification, and then using Image-Pro Plus software for statistical analysis. A calculation formula of an average surface particle diameter is ds=Σnidi3/Σnidi2, where ni represents the number of the metal particles with diameters being di, and the number of the selected metal particles is not less than 200. In the present disclosure, the particle size of the catalyst is small, the particle size of an active phase decreases, the number of chlorotrifluoroethylene and trifluoroethylene which are adsorbed at a local active site decreases, such that the coupling probability of the chlorotrifluoroethylene and the trifluoroethylene is reduced, the selectivity of the trifluoroethylene is improved, and deactivation of the catalyst caused by carbon deposition is effectively relieved. In another aspect, dissociated H formed around the main product trifluoroethylene is reduced, thereby facilitating desorption.

The disproportionation explosion of the trifluoroethylene during reaction is caused by the following three aspects: 1) trifluoroethylene polymerization initiates a disproportionation reaction; 2) high temperature and high pressure trigger the disproportionation reaction; and 3) chain propagation occurs in trifluoroethylene disproportionation, which reacts rapidly, causing a rapid rise in pressure and releasing a lot of heat.

By applying any one of the supported metal nano catalysts in the present disclosure to a reaction process of preparing the trifluoroethylene by means of hydrodechlorination of chlorotrifluoroethylene, the disproportionation reaction of the trifluoroethylene may be effectively inhibited, and the safety of the reaction is greatly enhanced. Specific findings are as follows: (1) By means of the supported metal nano catalyst of the present disclosure, the generation of trace HCFC-123a and/or HCFC-133 may be controlled, chlorine atoms in HCFC-123a or HCFC-133 molecules are used to capture intermediate radicals of the trifluoroethylene reaction to imped a chain propagation, so as to inhibit the occurrence of the disproportionation reaction of the trifluoroethylene. (2) By means of the supported metal nano catalyst of the present disclosure, the generation of a small amount of a by-product HFC-143 is controlled, and the disproportionation reaction of the trifluoroethylene is inhibited by means of a heat dilution effect of the HFC-143. (3) By means of the supported metal nano catalyst of the present disclosure, the conversion rate of the raw material chlorotrifluoroethylene is controlled to not be higher than 50%, thereby preventing hotspot from generating in a reactor, that is, avoiding triggering of the disproportionation reaction of the trifluoroethylene due to a high temperature.

The supported metal nano catalyst of the present disclosure is prepared by means of the following steps.

A1. Reduction and modification of a carrier: a reduction treatment is performed on the activated carbon carrier for 1.5-3 h at 200-800° C. by a reductant, and then cooling is performed to room temperature. The reductant is selected from at least one of hydrogen, nitrogen or ammonia.

A2. Nanoparticle deposition: a mixture of nanoparticle stabilizing agent, potassium bromide and potassium chloride is heated at 80-110° C. under stirring, and refluxing is performed for 1-2 h; then, a first component soluble salt and a second component soluble salt are added into the mixture, reaction is performed for 1.5-2.5 h by holding the temperature at 80-110° C., and then cooling is performed to room temperature to obtain a product; and excessive liquid phase reductant is dripped into the product under stirring, then the activated carbon carrier which is reduced and modified in step A1 is added, alkaline solutions is continuously dripped, a pH value is controlled to be 6-10.5, preferably the pH value is 9-10.5, and the metal nanoparticles are deposited on a surface of the activated carbon carrier.

A3. Washing and baking: filtration is performed, deoxidized deionized water or ethanol is used to perform washing to neutral, then drying is performed, and baking is performed for 1.0-4.0 h at 300-400° C. in an inert atmosphere, so as to obtain a catalyst precursor. Preferably, a baking temperature is about 350° C., and baking time is about 1.5-2.5 h.

A4. Reduction activation: the catalyst precursor is placed under a mixed atmosphere of hydrogen gas and nitrogen gas, the temperature is risen to 250-450° C. at the rate of 0.1-2.0° C./min, and a constant temperature is held for 1-5 hours, so as to obtain the supported metal nano catalyst.

In step A1, the reductant is preferably a combination of hydrogen gas and nitrogen gas, or a combination of ammonia and nitrogen gas. An acidic functional group of an activated carbon carrier after modification is decomposed, and the content of oxygen decreases; and at the same time, an alkaline functional group increases, and a nitrogen-containing functional group is coated on the surface of the carrier. The reduced and modified activated carbon carrier has enhanced adsorption capacity for non-polar substances.

In step A2, the first component soluble salt is selected from at least one of chloride, hydrochloride or organic salt of the first component, for example, ruthenium trichloride, ruthenium acetate, chloride or hydrochloride of palladium or platinum, an ethanol solution of sodium tetrachloropalladate (Na₂PdCl₄), ammonium chloroplatinate, or dipotassium tetrachloroplatinate (K₂PtCl₄). The second component soluble salt is selected from at least one of chloride, nitrate, sulfate or organic salt of the second component, for example, copper chloride, copper nitrate, copper sulfate, bismuth chloride or bismuth nitrate, ceric sulfate or cerium chloride heptahydrate.

In step A2, the nanoparticle stabilizing agent is selected from at least one of Polyvinylpyrrolidone (PVP), propylamine or Hexadecyl Trimethyl Ammonium Bromide (CTAB); and a molar dosage is 4-6 times of the sum of the molar weights of the first component and the second component.

In step A2, the liquid phase reductant is selected from at least one of L-ascorbic acid, NaBH₄, citric acid or ethylene glycol; and a molar dosage is 2-4 times of the sum of the molar weights of the first component and the second component.

In step A2, the alkaline solutions is a NaOH or KOH solution, and a mass concentration is 2-10 wt %.

In step A2, in a mixture of potassium bromide and potassium chloride, the mole ratio of the potassium chloride to the potassium bromide is 1:0.01-1:0.3.

The supported metal nano catalyst of the present disclosure includes a crystal face (111) and a crystal face (100). Through research, the applicant has found that, at different crystal plane proportions, the depth of the hydrogenation reaction of the catalyst of the chlorotrifluoroethylene and product distribution can be regulated and controlled. The crystal face (111) is conductive to producing a target product trifluoroethylene TrFE and producing trace HCFC-123a (a reaction path: CTFE→HCFC-123a→TrFE); and the crystal face (100) has stronger adsorption capacity for the chlorotrifluoroethylene, thereby facilitating the production of by-products HCFC-133 and HFC-143. In the present disclosure, by means of changing the ratio of the potassium bromide to the potassium chloride, the ratio of the crystal face (111) to the crystal face (100) may be regulated and controlled, thereby the depth of catalytic hydrogenation reaction and product distribution can be controlled. The reason lies in that, \textcircled{1} Br— preferentially adsorbs the crystal faces (100) of metal cations, that is, crystal growth is enhanced in a direction of (100). \textcircled{2} Compared to Br⁻ or Cr⁻ shows relatively weak interaction with the metal cations, and since the surface energy of a metal surface (111) is the lowest and nanoparticles try to minimize the total surface energy [note: (111)<(100)<(110)], the use of potassium chloride facilitates the generation of the crystal face (111) of the alloy.

In step A3, the nanoparticle stabilizing agent and the liquid phase reductant are removed by means of alcohol washing and baking, so as to prevent the nanoparticle stabilizing agent and the liquid phase reductant from adsorbing on the surface of the catalyst, which leads to a weakened contact between reactants and the active site, that is, it could cause a reduction in catalytic activity.

The present disclosure further provides a method for continuously producing 1,2-dibromo-trifluoroethane. The method includes: the obtained mixture entering a rectification apparatus for separation, trifluoroethylene obtained by means of rectification entering a second reactor and continuously reacting with bromine under light to obtain 1,2-dibromo-trifluoroethane; and the chlorotrifluoroethylene obtained by means of rectification returning to the first reactor for recycling.

The rectification apparatus includes a rectifying column with at least two stages. The trifluoroethylene is extracted from the top of the first-stage rectifying column, and the chlorotrifluoroethylene is extracted from a return tube of the last-stage rectifying column.

In a specific implementation, the two-stage rectifying column is used for the separation of the mixture. The temperature of a column bottom of the first-stage rectifying column is 20° C.-40° C., the temperature of a condenser is −10° C.-0° C., and the pressure of the column bottom is 0.5-1.5 MPa. The temperature of a column bottom of the second-stage rectifying column is 40-60° C., the temperature of the condenser is 0° C.-10° C., and the pressure of the column bottom is 0.3-0.8 MPa.

Due to the safety problem of the trifluoroethylene, the rectified trifluoroethylene needs to enter the second reactor immediately, and continuously reacts with bromine under light, so as to obtain the 1,2-dibromo-trifluoroethane. A complete reaction equation is shown as follows.

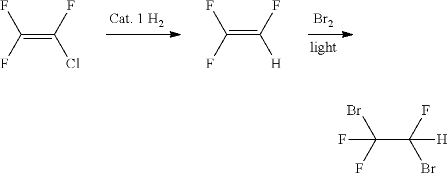

The bromine is bromine vapor; and the mole ratio of the trifluoroethylene to the bromine vapor is 1:(0.3-3). Preferably, the mole ratio of the trifluoroethylene to the bromine vapor is 1:(0.9-1.1).

A reaction temperature of the trifluoroethylene and the bromine vapor is 0° C.-150° C., and a pressure is 0-1 MPa. Preferably, the reaction temperature of the trifluoroethylene and the bromine vapor is 20° C.-80° C., and the pressure is 0-0.3 MPa.

A second portion of the present disclosure provides a method for continuously producing bromotrifluoroethylene. The production method includes: in a third reactor pre-loaded with 1,2-dibromo-trifluoroethane, continuously adding the 1,2-dibromo-trifluoroethane and solid alkali for reaction, so as to obtain bromotrifluoroethylene. A reaction equation is shown as follows.

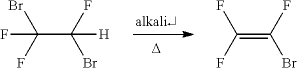

The method specifically includes the following reaction steps.

B1. The 1,2-dibromo-trifluoroethane (as a solvent) is added in the third reactor in advance, and an external circulating pump is opened, so as to cause the 1,2-dibromo-trifluoroethane to flow out from the third reactor and then return to the third reactor after passing through a filter apparatus.

B2. A temperature is risen to a reaction temperature, the 1,2-dibromo-trifluoroethane and the solid alkali are continuously added, a bromotrifluoroethylene gas is collected, bromotrifluoroethylene liquid is obtained after condensation, and by-products are discharged out of a reaction system via the filter apparatus, so as to achieve continuous reaction.

In B1 step, the volume of the 1,2-dibromo-trifluoroethane added in the third reactor in advance is ¼-½ of the volume of the third reactor. The external circulating pump has a flow rate of 1/5 to 5 times the volume of the third reactor per hour.

In B2 step, the 1,2-dibromo-trifluoroethane is continuously fed, and the feeding amount per hour is (0.01-0.1) of the amount added in advance.

The mole ratio of feeding rates of the solid alkali and the 1,2-dibromo-trifluoroethane is 1:(0.8-1.2). Preferably, the mole ratio of feeding rates of the solid alkali and the 1,2-dibromo-trifluoroethane is 1:1.

The temperature of the dehydrobromination reaction is 30-80° C., preferably, the temperature of the reaction is 60° C.-70° C.

The solid alkali is selected from at least one of lithium hydroxide, sodium hydroxide, potassium hydroxide, calcium hydroxide, sodium carbonate or potassium carbonate. Preferably, the solid alkali is selected from at least one of the sodium hydroxide, potassium hydroxide, the sodium carbonate or the potassium carbonate.

In order to enhance reaction efficiency, the particle size of the solid alkali ranges from 10 μm to 5 mm; and preferably, the particle size of the solid alkali ranges from 100 μm to 1 mm.

The bromotrifluoroethylene enters the condenser from a gas phase outlet of the third reactor for condensation and collection, and the temperature of the condenser is selected from −15° C. to −5° C.

A third portion of the present disclosure provides a method for producing hexafluoro-1,3-butadiene. The production method includes the following steps.

(1) The method for continuously producing trifluoroethylene is used to prepare a mixture comprising trifluoroethylene in a first reactor, and the mixture is used to prepare 1,2-dibromo-trifluoroethane in a second reactor by means of the method for continuously producing 1,2-dibromo-trifluoroethane.

(2) The method for continuously producing bromotrifluoroethylene is used to prepare bromotrifluoroethylene in a third reactor.

(3) The bromotrifluoroethylene is added to a fourth reactor holding with zinc powder, an initiator and an organic solvent for reaction, so as to obtain trifluoroethenyl zinc bromide, and the mixture enters a fifth reactor after filtration; and a coupling agent is added for a coupling reaction, so as to obtain hexafluoro-1,3-butadiene. A reaction equation is shown as follows.

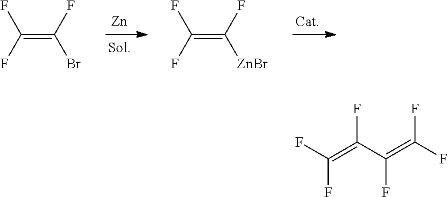

The organic solvent, the initiator and the zinc powder are first added to the fourth reactor, stirred and heated to 0-100° C. (preferably 30-60° C.), and then the bromotrifluoroethylene is added for reaction, so as to obtain a trifluoroethenyl zinc bromide solution; the zinc powder in the trifluoroethenyl zinc bromide solution is removed by means of filtration, then the mixture enters to the fifth reactor, and the coupling agent is added at −20-50° C. (preferably −10-10° C.) for reaction, so as to obtain the hexafluoro-1,3-butadiene.

The organic solvent is selected from at least one of N,N-dimethylformamide (DMF), N,N-dimethylacetamide (DMAc), Dimethyl Sulfoxide (DMSO) or Tetrahydrofuran (THF); and moisture contained in the organic solvent is ≤200 ppm.

The initiator is selected from at least one of methyl bromide, 1,2-dibromoethane, iodine, chlorotrimethylsilane or the trifluoroethenyl zinc bromide solution.

The coupling agent is selected from at least one of copper iodide, copper bromide, copper chloride, ferric chloride or ferric bromide.

A fourth portion of the present disclosure provides a system for producing hexafluoro-1,3-butadiene. The production system includes a subsystem X, a subsystem Y and a subsystem Z.

(1) The subsystem X for producing 1,2-dibromo-trifluoroethane includes a first reactor, a water-alkali washing apparatus, a rectification apparatus and a second reactor that are connected in order. The first reactor is a gas-solid phase reactor filled with the supported metal nano catalyst, and is provided with a raw material gas inlet and a mixture outlet. The mixture outlet communicates with an inlet of the rectification apparatus. A column top of the rectification apparatus is connected to a trifluoroethylene inlet of the second reactor. The second reactor is a photobromination reactor and is also provided with a bromine vapor inlet, a 1,2-dibromo-trifluoroethane outlet and a non-condensable gas outlet.

(2) The subsystem Y for producing bromotrifluoroethylene includes a third reactor. The third reactor is a dehydrobromination reactor, and is provided with a solid alkali continuous feeding apparatus, a 1,2-dibromo-trifluoroethane inlet connected to the 1,2-dibromo-trifluoroethane outlet of the second reactor, a discharging port and a bromotrifluoroethylene outlet. The discharging port is connected to a filter apparatus, so as to filter out by-products. The bromotrifluoroethylene outlet is successively connected to a condenser A and a condenser B. The condenser A is configured to return the 1,2-dibromo-trifluoroethane, and the condenser B is configured to condense the bromotrifluoroethylene.

(3) The subsystem Z for producing hexafluoro-1,3-butadiene includes a fourth reactor, a fifth reactor and a hexafluoro-1,3-butadiene collecting apparatus that are connected in order. The fourth reactor communicates with the bromotrifluoroethylene outlet of the third reactor.

It is known to those skilled in the art that, the apparatuses need to be connected to each other by means of pipelines; some valves and transfer pumps are disposed at inlets and outlets; and raw materials and products are usually stored by storage tanks (for example, a chlorotrifluoroethylene storage tank, a bromine storage tank, a bromotrifluoroethylene storage tank, and a hexafluoro-1,3-butadiene storage tank). The above are common knowledge and should be known to those skilled in the art, and are not described herein again.

Further, the rectification apparatus includes a first rectifying column and a second rectifying column. The mixture containing trifluoroethylene passes through the water-alkali washing apparatus and then enters the first rectifying column by means of compression (emptying excess hydrogen gas which is not compressed). The trifluoroethylene is collected from a column top and enters the second reactor, and remaining materials enter the second rectifying column. The chlorotrifluoroethylene is collected from a return tube and returns to the first reactor for recycling.

Further, the 1,2-dibromo-trifluoroethane after the filter apparatus filters out the by-products returns to the third reactor.

Further, an inlet of the fourth reactor is separately connected to an organic solvent feeding apparatus, a zinc powder feeding apparatus and a bromotrifluoroethylene feeding apparatus, and an outlet is connected to an excess zinc powder filter apparatus. An inlet of the fifth reactor is connected to an outlet of the zinc powder filter apparatus and is provided with a coupling agent feeding apparatus, and an outlet is connected to the hexafluoro-1,3-butadiene collecting apparatus.

The collecting apparatus includes a condenser and a storage tank.

A material of the first reactor is selected from one of 316L, Inconel 600 alloy, Monel 400 alloy or Hastelloy C alloy. A material of the second reactor is selected from one of silicate glass, quartz glass or silicon carbide. A material of the third reactor is selected from one of glass lining, silicon carbide or carbon steel lined with PTFE. A material of the fourth reactor is selected from one of the glass lining, the silicon carbide, the 316L or the carbon steel lined with PTFE. A material of the fifth reactor is selected from one of the glass lining, the silicon carbide, the 316L or the carbon steel lined with PTFE.

Compared with the prior art, the present disclosure has the following beneficial effects. 1. The supported metal nano catalyst of the present disclosure may control the by-products of the specific reaction, and eliminate an energy source or block a chain reaction so as to avoid the occurrence of the disproportionation reaction of the trifluoroethylene by a capturing behavior of the by-products HCFC-123a and HCFC-133 on trifluoroethylene radicals; and by means of the heat dilution effect of the by-product HFC-143, the conversion rate of the reaction is controlled to avoid the generation of hotspots, thereby achieving the safe production of the trifluoroethylene. 2. While the supported metal nano catalyst of the present disclosure maintains the low conversion rate of the chlorotrifluoroethylene, the catalyst still has long service life, good stability, and desirable carbon deposition resistance and sintering resistance. 3. In the present disclosure, by means of using the process of performing continuous dehydrobromination on the solid alkali, the contact between the product bromotrifluoroethylene and a proton-type solvent is avoided, such that production efficiency and reaction efficiency are enhanced, and the discharging of the three wastes is reduced.

### BRIEF DESCRIPTION OF THE DRAWINGS

FIG. 1a is a subsystem X of a 1,2-dibromo-trifluoroethane section in a system for producing hexafluoro-1,3-butadiene according to Embodiment VI of the present disclosure, including: 1: Chlorotrifluoroethylene cylinder, 2: Hydrogen gas cylinder, 3: First reactor, 4: Water-alkali washing apparatus, 5: Compressor, 6: Condenser, 7: First rectifying column, 8: Second rectifying column, 9: Second reactor, 10: Bromine evaporation tank, 11: 1,2-dibromo-trifluoroethane storage tank, 12: Chlorotrifluoroethylene storage tank.

FIG. 1b is a subsystem Y of a bromotrifluoroethylene section in a system for producing hexafluoro-1,3-butadiene according to Embodiment VI of the present disclosure, including: 13: 1,2-dibromo-trifluoroethane storage tank, 14: Third reactor, 15: Solid alkali storage tank, 16: Condenser, 17: Condenser, 18: Bromotrifluoroethylene storage tank, 19: By-product filter tank, 20: By-product filter tank.

FIG. 1c is a subsystem Z of a hexafluoro-1,3-butadiene section in a system for producing hexafluoro-1,3-butadiene according to Embodiment VI of the present disclosure, including: 21: Bromotrifluoroethylene cylinder, 22: Fourth reactor, 23: Zinc powder storage tank, 24: Condenser, 25: Zinc powder filter apparatus, 26: Fifth reactor, 27: Coupling agent storage tank, 28: Condenser, 29: Condenser, 30: Hexafluoro-1,3-butadiene storage tank.

FIG. 2 is a safety test curve of a Cat1 hydrogenation product prepared in Preparation example 1 of the present disclosure.

FIG. 3 is a safety test curve of a Cat-DB2 hydrogenation product prepared in Comparative preparation example 2 of the present disclosure.

### DETAILED DESCRIPTION OF THE EMBODIMENTS

The present disclosure is further described below with reference to specific embodiments, but the disclosure is not limited to these specific embodiments. It should be recognized by those skilled in the art that, the present disclosure covers all alternatives, improvements and equivalents that may be included within the scope of the claims.

### Preparation Example 1

This preparation example provided the preparation of a supported metal nano catalyst, specifically including the following steps.

A1. Reduction and modification of a carrier: 20 g of an activated carbon carrier was taken and placed in a mixed atmosphere (V_{NH3}:V_{N2}=1:5) of ammonia and nitrogen gas, treated for 2 h at 450° C., and cooled to room temperature for later use.

A2. Nanoparticle deposition: 7 mL of a hydrochloric acid solution of platinum chloride (Pt 3.8%), 0.6 g of BiCl₃ and 5 mL of a hydrochloric acid solution (with a concentration of 37%) were measured and dissolved in 40 mL of deionized water for later use; Polyvinylpyrrolidone (PVP) and a mixture of potassium chloride and potassium bromide (the mole ratio of KCl to KBr is 1:0.2) were placed in a round-bottomed flask, stirred in a magnetic manner, heated at 85° C., and refluxed for 1 h. Then a mixed solution of the platinum chloride and the BiCl₃ was added, heated for 2 h at 85° C., and cooled to room temperature; and excessive liquid phase reductant NaBH₄ was subsequently dripped. By means of maintaining a stirring state, the activated carbon carrier reduced and modified in step A1 was added, then a NaOH solution with the concentration being 3% was dripped, a pH value was controlled to be 9, and metal nanoparticles were deposited on a surface of the carrier.

A3. Washing and baking: filtration was performed, a filter cake was washed to neutral by deoxidized deionized water or ethanol, and then vacuum drying was performed for 8 h at 100° C. Then the filter cake was place into an atmosphere furnace, and baked for 2 h at 350° C. under a nitrogen atmosphere, and thus a catalyst precursor was obtained.

A4. Reduction activation: the catalyst precursor was placed under a mixed atmosphere (V_{hydrogen gas}·V_{nitrogen gas}=1:3) of hydrogen gas and nitrogen gas, the temperature was risen to 250° C. at the rate of 2.0° C./min, and a constant temperature was held for 2 hours, so as to obtain the supported metal nano catalyst, which was recorded as Cat1.

### Preparation Example 2

This preparation example provided the preparation of a supported metal nano catalyst, specifically including the following steps.

A1. The step of reducing and modifying the carrier was the same as Preparation example 1.

A2. Nanoparticle deposition: 6.1 mL of a hydrochloric acid solution of palladium chloride (the concentration being 0.033 g Pd/mL) and 2.3 g of Cu(NO₃)₂·3H₂O were dissolved in 40 mL of deionized water for later use; the PVP and the mixture of potassium chloride and potassium bromide (the mole ratio of KCl to KBr is 1:0.2) were placed in the round-bottomed flask, stirred in a magnetic manner, heated at 85° C., and refluxed for 1 h. Then a mixed solution of the palladium chloride and copper nitrate was added, heated for 2 h at 85° C., and cooled to room temperature; and an excessive liquid phase reductant NaBH₄ was subsequently dripped. By means of maintaining the stirring state, the activated carbon carrier reduced and modified in step A1 was added, then the NaOH solution with the concentration being 3% was dripped, the pH value was controlled to be 9.5, and the metal nanoparticles were deposited on the surface of the carrier.

A3. The step of washing and baking was the same as Preparation example 1.

A4. The step of reduction activation was the same as Preparation example 1, and the obtained supported metal nano catalyst was recorded as Cat2.

### Preparation Example 3

This preparation example provided the preparation of a supported metal nano catalyst, specifically including the following steps.

A1. The step of reducing and modifying the carrier was the same as Preparation example 1.

A2. Nanoparticle deposition: 0.7 g of ruodium (III) chloride hydrate (Ru 43%) and 3.1 g of Ce(NO₃)₃·6H₂O were measured and dissolved in 40 mL of deionized water for later use; Hexadecyl Trimethyl Ammonium Bromide (CTAB) and the mixture of potassium chloride and potassium bromide (the mole ratio of KCl to KBr is 1:0.2) were placed in the round-bottomed flask, stirred in a magnetic manner, heated at 85° C., and refluxed for 1 h. Then a mixed solution of the ruodium (III) chloride hydrate and cerium nitrate was added, heated for 2 h at 85° C., and cooled to room temperature; and an excessive liquid phase reductant NaBH₄ was subsequently dripped. By means of maintaining the stirring state, the activated carbon carrier reduced and modified in step A1 was added, then the NaOH solution with the concentration being 3% was dripped, the pH value was controlled to be 9.5, and the metal nanoparticles were deposited on the surface of the carrier.

A3. The step of washing and baking was the same as Preparation example 1.

A4. The step of reduction activation was the same as Preparation example 1, and the obtained supported metal nano catalyst was recorded as Cat3.

### Preparation Example 4

This preparation example provided the preparation of a supported metal nano catalyst, specifically including the following steps.

A1. The step of reducing and modifying the carrier was the same as Preparation example 1.

A2. Nanoparticle deposition: 5.2 g of the hydrochloric acid solution of platinum chloride (Pt 3.8%) and 1.3 g of Ce(NO₃)₃·6H₂O were dissolved in 40 mL of deionized water for later use; the CTAB and the mixture of potassium chloride and potassium bromide (the mole ratio of KCl to KBr was 1:0.2) are placed in the round-bottomed flask, stirred in a magnetic manner, heated at 85° C., and refluxed for 1 h; then a mixed solution of the platinum chloride and the cerium nitrate was added, heated for 2 h at 85° C., and cooled to room temperature; and an excessive liquid phase reductant NaBH₄ was subsequently dripped. By means of maintaining the stirring state, the activated carbon carrier reduced and modified in step A1 is added, then the NaOH solution with the concentration being 3% was dripped, the pH value was controlled to be 10, and the metal nanoparticles were deposited on the surface of the carrier.

A3. The step of washing and baking was the same as Preparation example 1.

A4. The step of reduction activation was the same as Preparation example 1, and the obtained supported metal nano catalyst was recorded as Cat4.

Preparation example 5 This preparation example provided the preparation of a supported metal nano catalyst, specifically including the following steps.

A1. The step of reducing and modifying the carrier was the same as Preparation example 1.

A2. Nanoparticle deposition: 6.1 mL of a hydrochloric acid solution of palladium chloride(the concentration being 0.033 g Pd/mL), 0.9 g of BiCl₃ and 5 mL of the hydrochloric acid solution (the concentration being 37%) were measured and dissolved in 40 mL of deionized water for later use; the PVP and the mixture of potassium chloride and potassium bromide (the mole ratio of KCl to KBr is 1:0.2) were placed in the round-bottomed flask, stirred in a magnetic manner, heated at 85° C., and refluxed for 1 h; then a mixed solution of the palladium chloride and the BiCl₃ was added, heated for 2 h at 85° C., and cooled to room temperature; and the excessive liquid phase reductant NaBH₄ was subsequently dripped. By means of maintaining the stirring state, the activated carbon carrier reduced and modified in step A1 was added, then the NaOH solution with the concentration being 3% was dripped, the pH value was controlled to be 10, and the metal nanoparticles were deposited on the surface of the carrier.

A3. The step of washing and baking was the same as Preparation example 1.

A4. The step of reduction activation was the same as Preparation example 1, and the obtained supported metal nano catalyst was recorded as Cat5.

### Comparative Preparation Example 1

This comparative preparation example provided the preparation of a supported metal nano catalyst, specifically including the following steps.

B1. The step of reducing and modifying the carrier was the same as Preparation example 1.

B2. Immersing: 0.6 g of the BiCl₃, 5 mL of the hydrochloric acid solution (38 wt %) and 7 mL of the hydrochloric acid solution of platinum chloride (Pt 3.8%) were weighed, 80.0 mL of distilled water was added for an uniform dilution, 20 g of the reduced and modified activated carbon carrier was added, immersing was performed for 2 h, and then drying was performed for 8 h at 110° C.

B3. Reduction activation: the catalyst precursor obtained in B1 step was placed under the mixed atmosphere of hydrogen gas and nitrogen gas, the temperature was risen to 250° C. at the rate of 2.0° C./min, and the constant temperature was held for 2 h, so as to obtain a hydrodechlorination catalyst Cat-DB1.

### Comparative Preparation Example 2

This comparative example provided the preparation of a supported metal nano catalyst. Specific operations are the same as that in Preparation example 1. The difference only lies in that, in step A2 of nanoparticle deposition, the potassium chloride was not used, only the potassium bromide was used, and a hydrodechlorination catalyst Cat-DB2 was obtained.

### Comparative Preparation Example 3

This comparative example provided the preparation of a supported metal nano catalyst. Specific operations were the same as that in Preparation example 1. The difference only lay in that, in step A2 of nanoparticle deposition, the mole ratio of the potassium chloride to the potassium bromide in the mixture was 0.2:1 and a hydrodechlorination catalyst Cat-DB3 was obtained.

### Embodiment 1

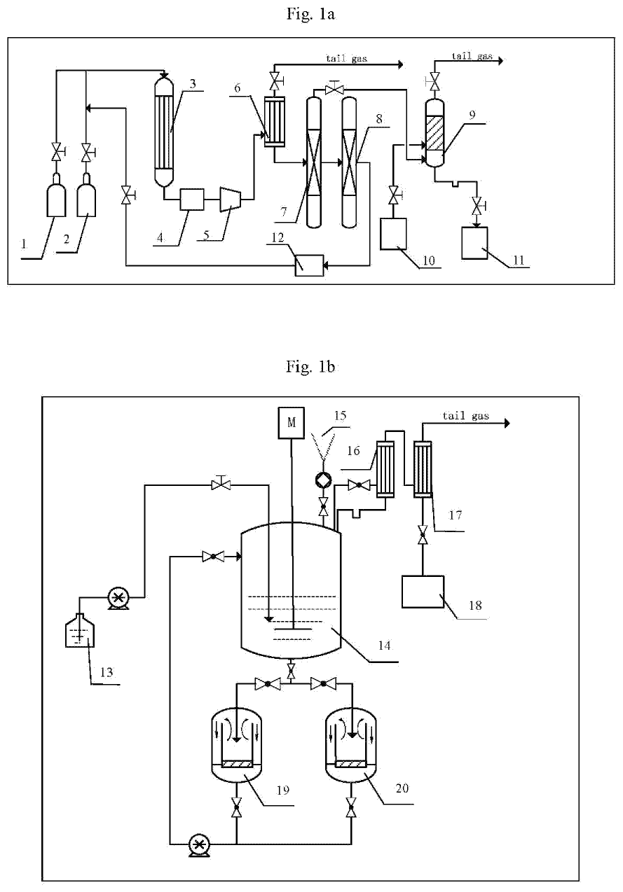

This embodiment provided a system for producing hexafluoro-1,3-butadiene. The production system included: a subsystem X of a 1,2-dibromo-trifluoroethane section shown in FIG. 1a, a subsystem Y of a bromotrifluoroethylene section shown in FIG. 1b and a subsystem Z of a hexafluoro-1,3-butadiene section shown in FIG. 1c.

Specifically, the subsystem X of the 1,2-dibromo-trifluoroethane section included a chlorotrifluoroethylene cylinder 1, a hydrogen gas cylinder 2, a first reactor 3, a water-alkali washing apparatus 4, a compressor 5, a condenser 6, a first rectifying column 7, a second rectifying column 8, a second reactor 9, a bromine evaporation tank 10, a 1,2-dibromo-trifluoroethane storage tank 11 and a chlorotrifluoroethylene storage tank 12.

The subsystem Y of the bromotrifluoroethylene section included a 1,2-dibromo-trifluoroethane storage tank 13, a third reactor 14, a solid alkali storage tank 15, a condenser 16, a condenser 17, a bromotrifluoroethylene storage tank 18, a by-product filter tank 19 and a by-product filter tank 20.

The subsystem Z of the hexafluoro-1,3-butadiene section included a bromotrifluoroethylene cylinder 21, a fourth reactor 22, a zinc powder storage tank 23, a condenser 24, a zinc powder filter apparatus 25, a fifth reactor 26, a coupling agent storage tank 27, a condenser 28, a condenser 29 and a hexafluoro-1,3-butadiene storage tank 30.

This embodiment further provided a method for producing hexafluoro-1,3-butadiene. The production method included the following steps.

S1. Preparation of trifluoroethylene and 1,2-dibromo-1,1,2-trifluoroethane: under the action of the supported metal nano catalyst, chlorotrifluoroethylene and hydrogen gas were subjected to a hydrodechlorination reaction in a first reactor, so as to obtain a mixture; the obtained mixture entered the first rectifying column and the second rectifying column for separation, the trifluoroethylene was collected from a column top of the first rectifying column, entered the second reactor, and continuously reacted with bromine under light, so as to obtain the 1,2-dibromo-trifluoroethane; and the chlorotrifluoroethylene extracted from a return tube of the second rectifying column returned to the first reactor for recycling.

S2. Preparation of bromotrifluoroethylene: the 1,2-dibromo-trifluoroethane, as a solvent, was added in the third reactor in advance, and an external circulating pump was opened, so as to cause the 1,2-dibromo-trifluoroethane to flow out from the third reactor and then return to the third reactor after passing through a filter apparatus; and a temperature was risen to a reaction temperature, the 1,2-dibromo-trifluoroethane and the solid alkali were continuously added, a bromotrifluoroethylene gas was collected, and bromotrifluoroethylene liquid was obtained after condensation.

S3. The bromotrifluoroethylene was added to the fourth reactor holding with zinc powder, an initiator and an organic solvent for reaction, so as to obtain trifluoroethenyl zinc bromide, and the mixture entered the fifth reactor after filtration; and the coupling agent was added for the coupling reaction, so as to obtain the hexafluoro-1,3-butadiene.

### Embodiment 1-1

Cat1 and Cat-DB2 were respectively used as the supported metal nano catalysts; the chlorotrifluoroethylene reacted with the hydrogen gas in the first reactor, the reaction temperature was 85° C., and the reaction pressure was the normal pressure; the total volume space velocity of the raw material hydrogen gas and the chlorotrifluoroethylene was 300 h⁻¹, and n_{(H2)}:n_{(CTFE)} was 2:1; and the mixture obtained by means of reaction was collected for safety test. A test method included: the test pressure environment of the mixture being 1.8 MPa, performing ignition at 30° C.

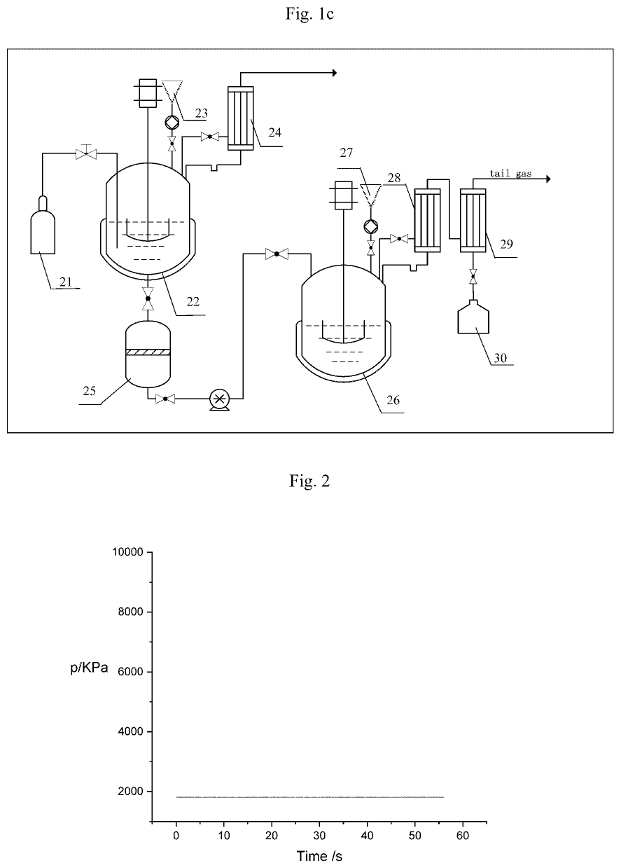

As shown in FIG. 2, when a hydrodechlorination product of the Cat1 was used for test, it was found that there was no obvious change in the reactor, that was, the trifluoroethylene was not exploded.

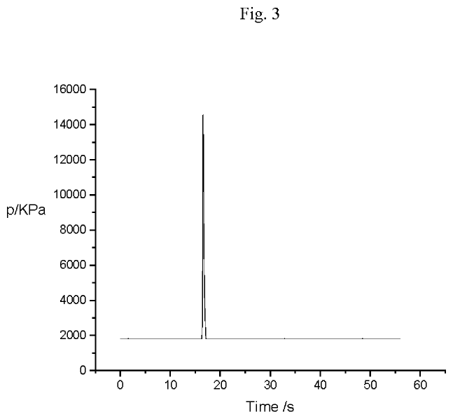

As shown in FIG. 3, when a hydrodechlorination product of the Cat-DB2 was used for test, it was found that the pressure in the reactor rapidly increases, that was, a TrFE mixture of the composition had the potential for disproportionation explosion.

### Embodiment 2-1

This embodiment provided preparation of the 1,2-dibromo-trifluoroethane, specifically including the following.

The subsystem X for producing the 1,2-dibromo-trifluoroethane was used. The first reactor was a fixed-bed reactor, with a material being Inconel 600 alloy, an internal diameter being 10 mm and a length being 550 mm; 10.0 g of Cat1-Cat5 and Cat-DB1-Cat-DB3 were respectively filled. The reaction temperature was 80-150° C., an operation pressure was the normal pressure, the raw material space velocity was 200-500 h⁻¹, and a raw material ratio was V_{H2}:V_{TrFE}=2:1. After water-alkali washing was performed on the reaction product, the reaction product was compressed and entered the first rectifying column (column bottom volume: 5 L, column diameter: 20 mm, column height: 3 m, column bottom temperature: 30° C., condenser temperature: −5° C., column bottom pressure: 0.8 MPa); and excess hydrogen gas which was not compressed was discharged from the top of the condenser. The mixed reaction gas was separated via the first rectifying column; and the trifluoroethylene was extracted from a gas phase on the top of the condenser of the first rectifying column, entered a photobromination reactor, and reacted with the bromine vapor under light (the mole ratio of the trifluoroethylene to the bromine being 1:0.95), so as to obtain the 1,2-dibromo-trifluoroethane. Materials at the column bottom of the first rectifying column entered the second rectifying column (column bottom volume: 5 L, column diameter: 20 mm, column height: 3 m, column bottom temperature: 50° C., condenser temperature: 0° C., column bottom pressure: 0.5 MPa); and the chlorotrifluoroethylene was collected to the storage tank from the return tube of the second rectifying column, and then entered a raw material supply pipeline of the first reactor via the storage tank.

The mixed reaction gases of different catalysts were sampled and analyzed before entering the first rectifying column, and results were shown in Table 1 below.

TABLE 1
Reaction evaluation results of different catalysts

| | | Raw | | | | |
|---|---|---|---|---|---|---|
| | Reaction | material | CTFE | TrFE | HFC-143 | R123a/R133 |
| | temperature/ | space | Conversion | Selectivity | Selectivity | Selectivity |
| Catalyst | ° C. | velocity/h⁻¹ | rate % | % | % | % |
| Cat 1 | 85 | 300 | 40 | 95.1 | 2.8 | 1.7 |
| Cat 2 | 90 | 200 | 48 | 94.0 | 4.4 | 1.2 |
| Cat 3 | 120 | 500 | 42 | 94.4 | 3.7 | 1.5 |
| Cat 4 | 150 | 470 | 49 | 94.7 | 3.8 | 1.0 |
| Cat 5 | 130 | 500 | 45 | 94.2 | 3.9 | 1.1 |
| Cat-DB1 | 85 | 300 | 90 | 56.1 | 39.0 | 3.7 |
| Cat-DB2 | 85 | 300 | 42 | 99.1 | — | 0.3 |
| Cat-DB3 | 85 | 300 | 45 | 10.5 | 86.2 | 2.6 |

When being separated via the first rectifying column, and before entering a photobromination reaction, results of sampling and analyzing the trifluoroethylene in each batch and results of analyzing the product after photobromination 1,2-dibromo-trifluoroethane were shown in the following table.

| Catalyst | Trifluoroethylene | 1,2-dibromo-trifluoroethane |
|---|---|---|
| Cat 1 | 99.2% | 98.6% |
| Cat 2 | 99.3% | 98.9% |
| Cat 3 | 99.3% | 99.0% |
| Cat 4 | 99.1% | 98.8% |
| Cat 5 | 99.5% | 99.1% |
| Cat-DB1 | 97.3% | 98.0% |
| Cat-DB2 | 99.2% | 98.7% |
| Cat-DB3 | 97.2% | 97.8% |

### Embodiment 3-1

This embodiment provided preparation of the bromotrifluoroethylene, specifically including the following.

Replacement was performed, by high-purity nitrogen, on the subsystem Y for producing the bromotrifluoroethylene, until oxygen content was ≤0.1%. 9600 kg (40 mol) of the 1,2-dibromo-trifluoroethane was added to a 10 L glass reactor, a heating apparatus with a stirrer and reactor jacket was opened, and the temperature in the reactor was controlled at 60° C.; inlet and outlet valves of a 1,2-dibromo-trifluoroethane condensing reflux device 16 were opened, and a jacket temperature was controlled at 5° C.; inlet and outlet valves of a bromotrifluoroethylene condensing reflux device 17 were opened, and the jacket temperature was controlled at −15° C.; a bottom valve of the reactor was opened, so as to communicate with a 2 L external filter apparatus; and an external circulating pump was opened, and a flow rate was controlled to be 2 L/h. A 1,2-dibromo-trifluoroethane feeding pump was opened, and a flow rate was controlled to be 400 g/h; a solid feeding device was opened, and sodium hydroxide granules were added to the reactor at 66.4 g/h. The 1,2-dibromo-trifluoroethane vapor returned to the reactor via the condenser 16, and the bromotrifluoroethylene was collected to a low-temperature freezer via the condenser 17. Continuous reaction was performed for 24 h; the total feeding amount of the 1,2-dibromo-trifluoroethane was 9600.5 g, the total feeding amount of the sodium hydroxide was 1600.5 g. A theoretical yield was 6414 g; and 6188.1 g of a product bromotrifluoroethylene was collected, which was anhydrous liquid, and had chromatographic purity of 98.5% and yield of 95.0% (calculated as sodium hydroxide).

### Embodiment 3-2

The operation of this embodiment was the same as that of Embodiment 3-1, and the difference lay in that, potassium hydroxide was used instead of the sodium hydroxide, a feeding rate was 93 g/h, and other conditions remained unchanged. The feeding amount and feeding rate of the 1,2-dibromo-trifluoroethane were the same. Continuous reaction was performed for 24 h; the total feeding amount of the 1,2-dibromo-trifluoroethane was 9600.5 g, the total feeding amount of the potassium hydroxide was 2232 g. A theoretical yield was 6414 g; and 6125.0 g of a product bromotrifluoroethylene was collected, which was anhydrous liquid, and had chromatographic purity of 98.2% and yield of 93.8% (calculated as potassium hydroxide).

### Embodiment 3-3

The operation of this embodiment was the same as that of Embodiment 3-1, and the difference lay in that, sodium carbonate was used instead of the sodium hydroxide, a feeding rate was 88 g/h, and other conditions remained unchanged. The feeding amount and feeding rate of the 1,2-dibromo-trifluoroethane were the same. Continuous reaction was performed for 24 h; the total feeding amount of the 1,2-dibromo-trifluoroethane was 9600.5 g, the total feeding amount of the sodium carbonate was 2112 g. A theoretical yield was 6414 g; and 5120.7 g of a product bromotrifluoroethylene was collected, which was anhydrous liquid, and had chromatographic purity of 98.2% and yield of 78.4% (calculated as sodium carbonate).

### Embodiment 3-4

The operation of this embodiment was the same as that of Embodiment 3-1, and the difference lay in that, the reaction temperature was reduced from original 60° C. to 50° C., and other conditions remained unchanged. Continuous reaction was performed for 24 h; the total feeding amount of the 1,2-dibromo-trifluoroethane was 9600.5 g, the total feeding amount of the sodium hydroxide was 1588.8 g. A theoretical yield was 6414 g; and 5355.8 g of a product bromotrifluoroethylene was collected, which was anhydrous liquid, and had chromatographic purity of 98.8% and yield of 82.5% (calculated as sodium hydroxide).

### Embodiment 3-5

The operation of this embodiment was the same as that of Embodiment 3-1, and the difference lay in that, the reaction temperature was increased from original 60° C. to 70° C., and other conditions remained unchanged. Continuous reaction was performed for 24 h; the total feeding amount of the 1,2-dibromo-trifluoroethane was 9600.5 g, the total feeding amount of the sodium hydroxide was 1588.8 g. A theoretical yield was 6414 g; and 6473.8 g of a product bromotrifluoroethylene was collected, which was anhydrous liquid, and had chromatographic purity of 96.6% and yield of 97.5% (calculated as sodium hydroxide).

### Embodiment 3-6

The operation of this embodiment was the same as that of Embodiment 3-1, and the difference lay in that, the feeding rate of the 1,2-dibromo-trifluoroethane was increased from original 400 g/h to 800 g/h, the feeding rate of the sodium hydroxide was increased from original 66.2 g/h to 132.4 g/h, and other conditions remained unchanged. Continuous reaction was performed for 12 h; the total feeding amount of the 1,2-dibromo-trifluoroethane was 9600.5 g, the total feeding amount of the sodium hydroxide was 1588.8 g. A theoretical yield was 6414 g; and 5971.0 g of a product bromotrifluoroethylene was collected, which was anhydrous liquid, and had chromatographic purity of 97.0% and yield of 90.3% (calculated as sodium hydroxide).

### Comparative Example 3-1

The operation of this embodiment was the same as that of Embodiment 3-1, and the difference lay in that, solid sodium hydroxide was replaced with a 30% sodium hydroxide solution, a feeding rate was 221 g/h, and other conditions remained unchanged. Continuous reaction was performed for 12 h; the total feeding amount of the 1,2-dibromo-trifluoroethane was 4800 g, the total feeding amount of the 30% sodium hydroxide solution was 2652 g. A theoretical yield was 3202.3 g; and 2495.0 g of a product bromotrifluoroethylene was collected, which was anhydrous liquid, and had chromatographic purity of 97.8% and yield of 76.2% (calculated as 30% sodium hydroxide solution).

### Comparative Example 3-2

Replacement was performed, by high-purity nitrogen, on the subsystem Y for producing the bromotrifluoroethylene, until oxygen content was 0.1%. 4000 g of the 30% sodium hydroxide solution (60 mol NaOH, 1.5 eq) was added to the 10 L glass reactor, the heating apparatus with a stirrer and reactor jacket was opened, and the temperature in the reactor was controlled at 60° C.; inlet and outlet valves of a 1,2-dibromo-trifluoroethane condensing reflux device A were opened, and a jacket temperature was controlled at 5° C.; inlet and outlet valves of a bromotrifluoroethylene condensing reflux device B were opened, and the jacket temperature was controlled at −15° C.; and 4800 kg (20 mol) of the 1,2-dibromo-trifluoroethane was added to the reactor, a feeding rate was 1200 g/h, feeding was completed within 4 hours, the temperature was held for 1 hour after feeding was completed, and then the reaction was stopped. A theoretical yield was 3220 g; and 2636.7 g of a product bromotrifluoroethylene was collected, which was anhydrous liquid, and had chromatographic purity of 95.5% and yield of 78.2% (calculated as 1,2-dibromo-trifluoroethane).

### Embodiment 4-1

This embodiment provided preparation of the hexafluoro-1,3-butadiene, specifically including the following.

Preparation of a trifluoroethenyl zinc bromide solution: replacement was performed, by high-purity nitrogen, on the subsystem Z for producing the hexafluoro-1,3-butadiene, until oxygen content was ≤0.1%. 3000 g of a N,N-dimethylformamide solution (moisture 150 ppm), 468 g (7.1 mol) of 300-mesh activated zinc powder and 300 g of a N,N-dimethylformamide solution of an initiator trifluoroethenyl zinc bromide were added to the fourth reactor (5 L glass reactor), and were heated to 45° C. under stirring; 886.2 g (5.5 mol) of bromotrifluoroethylene was continuously added for reaction, the temperature was held 45° C. for 1 hour after addition, and the reaction was completed. Then the mixture was left to stand for 3 hours, excess zinc powder and reaction solution were separated by means of filtration. The excess zinc powder was used indiscriminately after acid washing, water washing and vacuum drying. The reaction solution was transferred to the fifth reactor.

Preparation of the hexafluoro-1,3-butadiene: the fifth reactor holding the trifluoroethenyl zinc bromide solution was cooled to 0° C.; under stirring, 892.1 g (5.5 mol) of anhydrous ferric chloride was added to the reactor for reaction by a solid feeding device; and a feeding rate was controlled at 300 g/h, and the internal temperature was maintained within a range of 0-5° C. After addition, the temperature was risen to 140° C.; and the hexafluoro-1,3-butadiene was completely evaporated, and was then condensed and collected to low-temperature condensation.

An experimental result showed that, 404.0 g of a product was obtained. Through gas chromatographic analysis, the content of the hexafluoro-1,3-butadiene was 97.8%, theoretical yield was 445.5 g, and yield was 88.7%.

### Embodiment 4-2

The operation of this embodiment was the same as that of Embodiment 4-1, and the difference lay in that, the initiator trifluoroethenyl zinc bromide solution was replaced with iodine, the feeding amount was 42 g, and other conditions remained unchanged.

An experimental result showed that, 400.8 g of a product was obtained. Through gas chromatographic analysis, the content of the hexafluoro-1,3-butadiene was 96.6%, theoretical yield was 445.5 g, and yield was 86.9%.

### Embodiment 4-3

The operation of this embodiment was the same as that of Embodiment 4-1, and the difference lay in that, the solvent N,N-dimethylformamide was replaced with tetrahydrofuran, the dosage remained unchanged, and other conditions remained unchanged.

An experimental result showed that, 383.7 g of a product was obtained. Through gas chromatographic analysis, the content of the hexafluoro-1,3-butadiene was 95.8%, theoretical yield was 445.5 g, and yield was 82.5%.

### Embodiment 4-4

The operation of this embodiment was the same as that of Embodiment 4-1, and the difference lay in that, the coupling agent ferric chloride was replaced with copper chloride, the dosage was 739.8 g (5.5 mol), and other conditions remained unchanged.

An experimental result showed that, 409.6 g of a product was obtained. Through gas chromatographic analysis, the content of the hexafluoro-1,3-butadiene was 98.1%, theoretical yield was 445.5 g, and yield was 90.2%.

### Embodiment 4-5

The operation of this embodiment was the same as that of Embodiment 4-1, and the difference lay in that, the reaction temperature for preparing the trifluoroethenyl zinc bromide solution was changed from original 45° C. to 60° C., and other conditions remained unchanged.

An experimental result showed that, 386.3 g of a product was obtained. Through gas chromatographic analysis, the content of the hexafluoro-1,3-butadiene was 96.3%, theoretical yield was 445.5 g, and yield was 83.5%.

### Embodiment 4-6

The operation of this embodiment was the same as that of Embodiment 4-1, and the difference lay in that, the reaction temperature for preparing the hexafluoro-1,3-butadiene was changed from original (0-5° C.) to (5-10° C.), and other conditions remained unchanged.

An experimental result showed that, 397.7 g of a product was obtained. Through gas chromatographic analysis, the content of the hexafluoro-1,3-butadiene was 95.0%, theoretical yield was 445.5 g, and yield was 84.8%.

What is claimed is:

1. A method for continuously producing trifluoroethylene, comprising: under the action of a supported metal nano catalyst, chlorotrifluoroethylene and hydrogen gas being subjected to a hydrodechlorination reaction in a first reactor, so as to obtain a mixture, wherein the mixture comprises 0.8%-2.0% of 1,2-dichlorotrifluoroethane and/or 1-chloro-1,2,2-trifluoroethane; and the supported metal nano catalyst comprises a first component selected from at least one of ruthenium, palladium or platinum, a second component selected from at least one of copper, bismuth or cerium, and an activated carbon carrier.

2. The method for continuously producing trifluoroethylene according to claim 1, wherein the mixture further comprises: 20%-50% of trifluoroethylene, 43%-77% of chlorotrifluoroethylene, and 2%-5% of 1,1,2-trifluoroethane.

3. The method for continuously producing trifluoroethylene according to claim 1, wherein on the basis of the mass of a carrier of the catalyst, the supported amount of the first component is 0.05%-5.0%, the supported amount of the second component is 0.01%-3.0%, and a mass ratio of the first component to the second component is 1:(0.1-5);
a particle size of the supported metal nano catalyst is 2-50 nm, and metal particles with particle sizes being 2-10 nm account for more than 90%.

4. The method for continuously producing trifluoroethylene according to claim 1, wherein the supported metal nano catalyst is prepared by means of the following steps:
A1. reduction and modification of a carrier: performing a reduction treatment on the activated carbon carrier for 1.5-3 h at 200-800° C. by a reductant, and then performing cooling to room temperature, wherein the reductant is selected from at least one of hydrogen, nitrogen or ammonia;
A2. nanoparticle deposition: heating a mixture of nanoparticle stabilizing agent, potassium bromide and potassium chloride to 80-110° C. under stirring, and performing refluxing for 1-2 h; then, adding a first component soluble salt and a second component soluble salt into the mixture, performing reaction for 1.5-2.5 h by holding the temperature at 80-110° C., and then performing cooling to room temperature to obtain a product; and dripping excessive liquid phase reductant into the product under stirring, then adding the activated carbon carrier which is reduced and modified in step A1, continuously dripping alkaline solutions, controlling a pH value to be 6-10.5, and depositing the metal nanoparticles on a surface of the activated carbon carrier;
A3. washing and baking: performing filtration, using deoxidized deionized water or ethanol to perform washing to neutral, then performing drying, and performing baking for 1.0-4.0 h at 300-400° C. in an inert atmosphere, so as to obtain a catalyst precursor; and
A4. reduction activation: placing the catalyst precursor under a mixed atmosphere of hydrogen gas and nitrogen gas, rising the temperature to 250-450° C. at the rate of 0.1-2.0° C./min, and holding a constant temperature for 1-5 hours, so as to obtain the supported metal nano catalyst.

5. The method for continuously producing trifluoroethylene according to claim 4, wherein in step A2, the first component soluble salt is selected from at least one of chloride, hydrochloride or organic salt of the first component; and the second component soluble salt is selected from at least one of chloride, nitrate, sulfate or organic salt of the second component;
the nanoparticle stabilizing agent is selected from at least one of Polyvinylpyrrolidone, polyacrylate or Hexadecyl Trimethyl Ammonium Bromide; and a molar dosage is 4-6 times of the sum of the molar weights of the first component and the second component;
the liquid phase reductant is selected from at least one of L-ascorbic acid, NaBH₄, citric acid or ethylene glycol; and a molar dosage is 2-4 times of the sum of the molar weights of the first component and the second component;
the alkaline solutions is a NaOH solution or KOH solution, and a mass concentration is 2-10 wt %;
in a mixture of potassium bromide and potassium chloride, the mole ratio of the potassium chloride to the potassium bromide is 1:0.01-1:0.3.

6. The method for continuously producing trifluoroethylene according to claim 1, wherein a reaction temperature of the chlorotrifluoroethylene and the hydrogen gas is 100-200° C., and a reaction pressure is 0-2 MPa; the raw material volume space velocity of the hydrogen gas and the chlorotrifluoroethylene is 200-500 h⁻¹; and the mole ratio of the hydrogen gas and the chlorotrifluoroethylene is (1.2-2.5):1.

7. A method for continuously producing 1,2-dibromo-trifluoroethane, wherein the mixture according to claim 1 enters a rectification apparatus for separation, trifluoroethylene obtained by means of rectification enters a second reactor and continuously reacts with bromine under light to obtain 1,2-dibromo-trifluoroethane; and the chlorotrifluoroethylene obtained by means of rectification returns to the first reactor for recycling.

8. The method for continuously producing 1,2-dibromo-trifluoroethane according to claim 7, wherein the rectification apparatus comprises a rectifying column with at least two stages, wherein the trifluoroethylene is extracted from the top of the first-stage rectifying column, and the chlorotrifluoroethylene is extracted from a return tube of the last-stage rectifying column.

9. The method for continuously producing 1,2-dibromo-trifluoroethane according to claim 7, wherein the bromine is bromine vapor; the mole ratio of the trifluoroethylene to the bromine vapor is 1:(0.3-3); and a reaction temperature of the trifluoroethylene and the bromine vapor is 0° C.-150° C., and a pressure is 0-1 MPa.

10. A method for continuously producing bromotrifluoroethylene, comprising: in a third reactor pre-loaded with 1,2-dibromo-trifluoroethane, continuously adding the 1,2-dibromo-trifluoroethane and solid alkali in the third reactor, and performing a dehydrobromination reaction to obtain bromotrifluoroethylene.

11. The method for continuously producing bromotrifluoroethylene according to claim 10, wherein the solid alkali is selected from at least one of lithium hydroxide, sodium hydroxide, potassium hydroxide, calcium hydroxide, sodium carbonate or potassium carbonate;
the mole ratio of feeding rates of the solid alkali and the 1,2-dibromo-trifluoroethane is 1:(0.8-1.2); and the temperature of the dehydrobromination reaction is 30-80° C.;
the volume of the pre-loaded 1,2-dibromo-trifluoroethane is 1/4-1/2 of the volume of the third reactor.

12. The method for continuously producing bromotrifluoroethylene according to claim 10, comprising the following steps:
B1. adding the 1,2-dibromo-trifluoroethane as a solvent in the third reactor in advance, and opening an external circulating pump, so as to cause the 1,2-dibromo-trifluoroethane to flow out from the third reactor and then return to the third reactor after passing through a filter apparatus; and
B2. rising a temperature to a reaction temperature, continuously adding the 1,2-dibromo-trifluoroethane and the solid alkali, collecting a bromotrifluoroethylene gas, obtaining bromotrifluoroethylene liquid after the bromotrifluoroethylene gas is condensed, and discharging by-products out of a reaction system via the filter apparatus, so as to achieve continuous reaction.

13. A method for producing hexafluoro-1,3-butadiene, comprising the following steps:
(1) using the method for continuously producing trifluoroethylene according to claim 1 to prepare a mixture comprising trifluoroethylene in a first reactor, and using the mixture to prepare 1,2-dibromo-trifluoroethane in a second reactor by means of the method for continuously producing 1,2-dibromo-trifluoroethane according to claim 7;
(2) using the method for continuously producing bromotrifluoroethylene according to claim 7 to prepare bromotrifluoroethylene in a third reactor; and
(3) adding the bromotrifluoroethylene to a fourth reactor holding with zinc powder, an initiator and an organic solvent for reaction, so as to obtain trifluoroethenyl zinc bromide, and the trifluoroethenyl zinc bromide entering a fifth reactor after filtration; and adding a coupling agent into the trifluoroethenyl zinc bromide for a coupling reaction, so as to obtain hexafluoro-1,3-butadiene.

14. The method for producing hexafluoro-1,3-butadiene according to claim 13, wherein the organic solvent, the initiator and the zinc powder are first added to the fourth reactor, stirred and heated to 0-100° C., and then the bromotrifluoroethylene is added in the fourth reactor for reaction, so as to obtain a trifluoroethenyl zinc bromide solution; the zinc powder in the trifluoroethenyl zinc bromide solution is removed by means of filtration, and the coupling agent is added into the trifluoroethenyl zinc bromide solution after filtration at −20-50° C. for reaction, so as to obtain the hexafluoro-1,3-butadiene.

15. The method for producing hexafluoro-1,3-butadiene according to claim 13, wherein the organic solvent is selected from at least one of N,N-dimethylformamide, N,N-dimethylacetamide, Dimethyl Sulfoxide or Tetrahydrofuran; and moisture contained in the organic solvent is 200 ppm;
the initiator is selected from at least one of methyl bromide, 1,2-dibromoethane, iodine, chlorotrimethylsilane or the trifluoroethenyl zinc bromide solution;
the coupling agent is selected from at least one of copper iodide, copper bromide, copper chloride, copper sulfate, copper acetate, ferric chloride or ferric bromide.

16. A system for producing hexafluoro-1,3-butadiene, comprising:
(1) a subsystem X for producing 1,2-dibromo-trifluoroethane, comprising a first reactor, a water-alkali washing apparatus, a rectification apparatus and a second reactor that are connected in order, wherein the first reactor is a gas-solid phase reactor filled with the supported metal nano catalyst according to claim 1, and is provided with a raw material gas inlet and a mixture outlet; the mixture outlet communicates with an inlet of the rectification apparatus; a column top of the rectification apparatus is connected to a trifluoroethylene inlet of the second reactor; and the second reactor is a photobromination reactor and is also provided with a bromine vapor inlet, a 1,2-dibromo-trifluoroethane outlet and a non-condensable gas outlet;
(2) a subsystem Y for producing bromotrifluoroethylene, comprising a third reactor, wherein the third reactor is a dehydrobromination reactor, and is provided with a solid alkali continuous feeding apparatus, a 1,2-dibromo-trifluoroethane inlet connected to the 1,2-dibromo-trifluoroethane outlet of the second reactor, a discharging port and a bromotrifluoroethylene outlet; the discharging port is connected to a filter apparatus, so as to filter out by-products; the bromotrifluoroethylene outlet is successively connected to a condenser A and a condenser B; and the condenser A is configured to return the 1,2-dibromo-trifluoroethane, and the condenser B is configured to condense the bromotrifluoroethylene; and
(3) a subsystem Z for producing hexafluoro-1,3-butadiene, comprising a fourth reactor, a fifth reactor and a hexafluoro-1,3-butadiene collecting apparatus that are connected in order, wherein the fourth reactor communicates with the bromotrifluoroethylene outlet of the third reactor.

17. The system for producing hexafluoro-1,3-butadiene according to claim 16, wherein the rectification apparatus comprises a first rectifying column and a second rectifying column; the mixture containing trifluoroethylene passes through the water-alkali washing apparatus and then enters the first rectifying column by means of compression, emptying excess hydrogen gas which is not compressed; the trifluoroethylene is collected from a column top and enters the second reactor, and remaining materials enter the second rectifying column; and the chlorotrifluoroethylene is collected from a return tube and returns to the first reactor for recycling.

18. The system for producing hexafluoro-1,3-butadiene according to claim 16, wherein the 1,2-dibromo-trifluoroethane after the filter apparatus filters out the by-products returns to the third reactor.

19. The system for producing hexafluoro-1,3-butadiene according to claim 16, wherein an inlet of the fourth reactor is separately connected to an organic solvent feeding apparatus, a zinc powder feeding apparatus and a bromotrifluoroethylene feeding apparatus, and an outlet is connected to an excess zinc powder filter apparatus; an inlet of the fifth reactor is connected to an outlet of the zinc powder filter apparatus and is provided with a coupling agent feeding apparatus, and an outlet is connected to the hexafluoro-1,3-butadiene collecting apparatus; and the collecting apparatus comprises a condenser and a storage tank.

20. The system for producing hexafluoro-1,3-butadiene according to claim 16, wherein a material of the first reactor is selected from one of 316 L, Inconel 600 alloy, Monel 400 alloy or Hastelloy C alloy; a material of the second reactor is selected from one of silicate glass, quartz glass or silicon carbide; a material of the third reactor is selected from one of glass lining, silicon carbide or carbon steel lined with PTFE; a material of the fourth reactor is selected from one of the glass lining, the silicon carbide, the 316 L or the carbon steel lined with PTFE; and a material of the fifth reactor is selected from one of the glass lining, the silicon carbide, the 316 L or the carbon steel lined with PTFE.
